SEQUENCE LISTING (3 sequences)

SEQ ID NO: 1 - protein, 6 aa, synthetic construct
CDWLPK

SEQ ID NO: 2 - protein, 5 aa, synthetic construct
DWLPK

SEQ ID NO: 3 - protein, 4 aa, synthetic construct
DWLP

(19) United States
(12) Patent Application Publication
(10) Pub. No.: US 2025/0041379 A1
(43) Pub. Date: Feb. 6, 2025

(54) METHODS AND COMPOSITIONS FOR THE TREATMENT OF CANCER

(71) Applicant: Vigeo Therapeutics, Inc., Cambridge, MA (US)

(72) Inventor: Jing Watnick, Cambridge, MA (US)

(73) Assignee: Vigeo Therapeutics, Inc., Cambridge, MA (US)

(21) Appl. No.: 18/717,178

(22) PCT Filed: Sep. 29, 2022
(86) PCT No.: PCT/US2022/045154

§ 371 Date: Jun. 6, 2024

Related U.S. Application Data
(60) Provisional application No. 63/290,834, filed on Dec. 17, 2021.

(51) Int. Cl. A61K 38/12; A61P 35/00; G01N 33/68

(52) U.S. Cl. CPC A61K 38/12; A61P 35/00; G01N 33/6893; G01N 2333/70596; G01N 2800/52

(57) ABSTRACT

The standard of care for cancer patients comprises administration of broadly cytotoxic agents with no direct anti-tumor activity. Such treatments present with debilitating side effects and are prone to the development of resistance. Accordingly, there is a need for improved cancer therapeutics that are directly anti-tumorigenic and antimetastatic. The present technology relates to methods and compositions for improved cancer therapy. In particular, the present technology relates to determining the levels of CD36 and CD47 in a cancer and administering agents that modulate their activities, wherein the cancer demonstrates elevated CD36 and CD47 levels or activity.

### CROSS-REFERENCE TO RELATED APPLICATIONS

This application claims priority to and the benefit of U.S. Provisional Application No. 63/290,834, filed Dec. 17, 2021, the entire contents of which are hereby incorporated by reference.

### TECHNICAL FIELD

The present technology relates to methods and compositions for improved cancer therapy. In particular, the present technology relates to determining the levels of CD36 and CD47 in a cancer and administering agents that modulate their activities, wherein the cancer demonstrates elevated CD36 and CD47 levels or activity.

### BACKGROUND

The standard of care for cancer patients comprises administration of broadly cytotoxic agents with no direct anti-tumor activity. Such treatments present with debilitating side effects and are prone to the development of resistance. Accordingly, there is a need for improved cancer therapeutics that are directly anti-tumorigenic and anti-metastatic.

One of the major barriers to the effective treatment of cancer is the immunosuppressive nature of the tumor microenvironment (TME), which is mediated by myeloid derived suppressor cells (MDSCs), M2 macrophages, and regulatory T cells (Tregs). High levels of the cell surface receptors CD36 and CD47 are associated with poor prognostic outcome for many types of cancers (Enciu et al., 2018; Huang et al., 2020), including pancreatic cancer and glioblasoma. However, there are currently known therapeutic agents that target both molecules simultaneously.

It has been previously shown that thrombodspondin-1 (TSP-1) binds to CD36 and CD47 to induce apoptosis in tumor and endothelial cells, increases the M1: M2 macrophage ratio, and activates cytotoxic T lymphocytes (CTLs) (Dawson et al 1996, Martin-Manso et al 2008, Russell et al. 2015). Accordingly, therapeutic agents that induce TSP-1will be useful in the treatment of cancers associated with dually elevated levels of CD36 and CD47.

### SUMMARY

In one aspect, the present disclosure provides a method for evaluating the responsiveness of a subject to cancer treatment with a Tsp-1-inducing agent, the method comprising determining levels of CD36 and CD47 in a sample obtained from the subject, wherein dually elevated levels of CD36 and CD47 in the sample compared to control levels indicates that the subject is or is likely to be responsive to cancer treatment with a Tsp-1-inducing agent.

In some embodiments, the levels of CD36 and CD47 in the sample are determined in vitro. In some embodiments, the method further comprises selecting the subject with dually elevated levels of CD36 and CD47 in the sample compared to control levels for treatment with a Tsp-1-inducing agent. In some embodiments, the method further comprises administering to the subject an effective amount of a Tsp-1-inducing agent to treat the cancer.

In one aspect, the present disclosure provides a method for treating a subject with cancer, the method comprising determining levels of CD36 and CD47 in a sample obtained from the subject, wherein dually elevated levels of CD36 and CD47 in the sample compared to control levels indicates that the subject is or is likely to be responsive to cancer treatment with a Tsp-1-inducing agent, and further comprising administering to a subject having dually elevated levels of CD36 and CD47 in the sample compared to control levels an effective amount of a Tsp-1-inducing agent to treat the cancer.

In one aspect, the present disclosure provides a method for treating a subject with cancer, the method comprising (a) selecting a subject with cancer on the basis that the subject is known to have dually elevated levels of CD36 and CD47 in a sample compared to control levels; and (b) administering to the subject an effective amount of a Tsp-1-inducing agent to treat the cancer.

In some embodiments, the control levels are CD36 and CD47 levels in a non-cancerous cell or tissue obtained from the subject. In some embodiments, the control levels are CD36 and CD47 levels in a cell or tissue obtained from a healthy subject or a population of healthy subjects. In some embodiments, the control levels are predetermined levels. In some embodiments, the levels of CD36 and CD47 comprise CD36 and CD47 protein levels a CD36 and CD47 mRNA levels. In some embodiments, the cancer is prostate cancer, breast cancer, ovarian cancer, lung cancer, leukemia, pancreatic cancer, glioblastoma multiforme, astrocytoma, or melanoma.

In some embodiments, the Tsp-1-inducing agent comprises a Psap peptide having the amino acid sequence CDWLPK (SEQ ID NO 1), DWLPK (SEQ ID NO 2), or DWLP (SEQ ID NO 3), or an amino acid substitution variant thereof, wherein the amino acid substitution is:
a) Tyrosine (Y) for Tryptophan (W);
b) an amino acid substitution for Leucine (L) selected from Valine (V), Alanine (A) or Glycine (G), or a non-canonical amino acid of similar size, or a derivative thereof; c) Arginine (R) for Lysine (K);
d) a D-isomer of Aspartic Acid (D) for an L-isomer of Aspartic Acid (D) and/or a D-isomer of Leucine (L) for a L-isomer of Leucine (L);
e) a D-isomer of Tryptophan (W) for an L-isomer of Tryptophan (W) and/or a D-isomer of Proline (P) for an L-isomer of Proline (P); or combinations thereof.

In some embodiments, the Psap peptide is 50 amino acids or fewer in length. In some embodiments, the Psap peptide is 30 amino acids or fewer in length. In some embodiments, the Psap peptide is 15 amino acids or fewer in length. In some embodiments, the Psap peptide is 6 amino acids or fewer in length. In some embodiments, the Psap peptide is a cyclic peptide. In some embodiments, the non-canonical amino acid of similar size is methylvaline, methylleucine, or sarcosine. In some embodiments, the Tsp-1-inducing agent is cyclic DWLPK (SEQ ID NO 2).

In one aspect, the present disclosure provides a composition for use in treating a subject with cancer characterized by dually elevated levels of CD36 and CD47 in a sample compared to control levels, the composition comprising a Tsp-1-inducing agent.

In one aspect, the present disclosure provides use of a composition for in the manufacture of a medicament for treating a subject with cancer characterized by dually elevated levels of CD36 and CD47 in a sample compared to control levels, the composition comprising a Tsp-1-inducing agent.

In some embodiments, the control levels are CD36 and CD47 levels in a non-cancerous cell or tissue obtained from the subject. In some embodiments, the control levels are CD36 and CD47 levels in a cell or tissue obtained from a healthy subject or a population of healthy subjects. In some embodiments, the control level is a predetermined level. In some embodiments, the levels of CD36 and CD47 comprise CD36 and CD47 protein levels a CD36 and CD47 mRNA levels. In some embodiments, the cancer is prostate cancer, breast cancer, ovarian cancer, lung cancer, leukemia, pancreatic cancer, glioblastoma multiforme, astrocytoma, or melanoma.

In some embodiments, the Tsp-1-inducing agent comprises a Psap peptide having the amino acid sequence CDWLPK, DWLPK, or DWLP, or an amino acid substitution variant thereof, wherein the amino acid substitution is:
a) Tyrosine (Y) for Tryptophan (W);
b) an amino acid substitution for Leucine (L) selected from Valine (V), Alanine (A) or Glycine (G), or a non-canonical amino acid of similar size, or a derivative thereof; c) Arginine (R) for Lysine (K);
d) a D-isomer of Aspartic Acid (D) for an L-isomer of Aspartic Acid (D) and/or a D-isomer of Leucine (L) for a L-isomer of Leucine (L);
e) a D-isomer of Tryptophan (W) for an L-isomer of Tryptophan (W) and/or a D-isomer of Proline (P) for an L-isomer of Proline (P); or combinations thereof.

In some embodiments, the Psap peptide is 50 amino acids or fewer in length. In some embodiments, the Psap peptide is 30 amino acids or fewer in length. In some embodiments, the Psap peptide is 15 amino acids or fewer in length. In some embodiments, the Psap peptide is 6 amino acids or fewer in length. In some embodiments, the Psap peptide is a cyclic peptide. In some embodiments, the non-canonical amino acid of similar size is methylvaline, methylleucine, or sarcosine. In some embodiments, the Tsp-1-inducing agent is cyclic DWLPK (SEQ ID NO 2). In some embodiments, the sample is a tumor sample.

### BRIEF DESCRIPTION OF THE FIGURES

FIG. 1 is a chart illustrating VT1021 modulation of the tumor microenvironment (TME) via CD36 and CD47. VT1021 binds to its receptor on myeloid derived suppressor cells (MDSC) and activates a signal transduction pathway culminating in the increased expression of thrombospondin-1 (Tsp-1). Tsp-1 then carries out a myriad of anti-tumor activities via binding to its two major cell surface receptors, CD36 and CD47.

FIG. 2 is a chart showing the pharmacokinetic profile of VT1021 concentration in the plasma of human patients with glioblastoma (red), pancreatic cancer (green), ovarian cancer (blue), other (purple) or not reported (red) over time following the initial administration at time 0. “Others” and “Not reported” refer to subjects enrolled in the clinical trial with solid tumor indications other than GBM, Panc, and Ovarian cancers.

FIG. 3 shows representative images of immunohistochemical staining of CD36 and CD47 depicting the characterization of high, medium, and low staining intensity.

FIG. 4 is a bar graph depicting the correlation between CD36 and CD47 expression in patient tumor tissue, as measured by immunohistochemistry staining, and the length of time each patient remained on study.

FIG. 5A and FIG. 5B are charts showing that VT1021 modulates the TME in pancreatic cancer. FIG. 5A shows metal ion immunostaining of overall tissue structure based on Vimentin, Keratin, double stranded (ds) DNA), thrombospondin-1, CD11b, and keratin staining in MDSCs, levels of monocytic MDSCs based on CD14, CD11b and keratin, cytotoxic T cells (CTLs) based on CD3 and CD8, and regulatory T cells (Tregs) based on CD3, and FoxP3 in patient tumor tissue before (pre) and during treatment with VT1021. FIG. 5B shows (left panel) metal ion immunostaining to identify and quantitate M1 macrophages using cell surface markers iNOS and CD68 and M2 macrophages using cell surface markers CD163 and CD68 in patient tumor tissue samples obtained prior to treatment with VT1021 (pre) and during treatment with VT1021 (On); (right panel) Graphical depiction of Tsp-1 protein levels, ratio of CTL to Tregs, ratio of M1: M2 macrophages and the fold change in macrophage subtype prior to and during treatment with VT1021 as measured by metal ion immunostaining.

FIG. 6 is a chart shows immunohistochemical staining of CD36 and CD47 protein expression in a tumor tissue microarray comprised of tissue from patients with pancreatic cancer.

FIG. 7 is a chart showing the pharmacokinetic profile of VT1021 concentration in the plasma of male (solid line) and female (dotted line) glioblastoma patients over time following the initial administration at time 0.

FIG. 8 is a swimmers plot depicting days on study for glioblastoma patients treated with VT1021, the colors of the bars indicate the patients expression levels of CD36 and CD47 as determined by immunohistochemical analysis of tumor tissue collected prior to the onset of treatment with VT1021, purple bars indicate high expression of both proteins, light blue bars indicate that either CD36, CD47, or neither had high expression, and gray bars indicate that the expression levels were not able to be determined.

FIG. 9 is a chart showing that VT1021 induces a complete response in rGBM subjects. (A) MRI images of a glioblastoma lesion in a patient treated with VT1021 depicting the decrease in lesion size over time during treatment; (B) graphical depiction of the change in the area of the lesion measured using MRI imaging over time during treatment with VT1021; (C) Immunohistochemical staining of CD36 and CD47 levels in this patient demonstrating high levels of expression of each protein.

FIG. 10 is a chart showing that VT1021 induces TSP-1 in the circulation and the TME. (A) Bar graph of Tsp-1 protein levels measured by ELISA in circulating MDSCs collected prior to (baseline) and after (induction) treatment with VT1021 from patients who experienced complete or partial response, stable disease, or progressive disease; (B) H&E and immunohistochemical analysis of patient tumor tissue prior to onset (pre) or during treatment with VT1021; (C) Immunohistochemical analysis depicting representative images of high levels of CD36 and CD47.

FIG. 11A, FIG. 11B, FIG. 11C, FIG. 11D, FIG. 11E, and FIG. 11F are charts demonstrating modulation of the immune system by VT1021 in circulation and in the TME.

FIG. 11A shows box plots of fold change in: (Upper Right) proliferating (Ki67+) cytotoxic T lymphocytes; (Upper Left) proliferating (Ki67+) Helper (CD4+) T lymphocytes; (Lower Right) monocytic (CD11b/CD14+) MDSCs; and (Lower Left) activated MDSCs; in patients who experienced complete or partial response (CR/PR), stable disease (SD), or progressive disease (PD) following treatment with VT1021.

FIG. 11B shows metal ion immunostaining of iNOS, CD68, and DNA to identify M1 macrophages and CD163, CD68, and DNA to identify M2 macrophages in patient tumor tissue.

FIG. 11C is a bar graph of the change in the percentage of M1 and M2 macrophages in patient tumor tissue as determined by metal ion immunostaining.

FIG. 11D shows metal ion immunostaining patient tumor tissue to examine overall tissue structure based on Vimentin, CD56, and double stranded (ds) DNA); levels of cytotoxic T cells (CTLs) based on CD3, CD8, and CD56; total levels of MDSCs based on CD11b and CD56; and levels of monocytic MDSCs based on CD11b, CD14, and CD56.

FIG. 11E is a bar graph depicting the fold-change in cytotoxic T lymphocytes (CTLs) in patients prior to the onset of treatment with VT1021 (pre-treatment) and during treatment with VT1021 (On-Study) as determined by metal ion immunostaining.

FIG. 11F is a bar graph depicting the fold-change in total and monocytic myeloid derived suppressor cells (MDSCs) in patients prior to the onset of treatment with VT1021 (pre-treatment) and during treatment with VT1021 (On-Study) as determined by metal ion immunostaining.

### DETAILED DESCRIPTION

Provided herein are novel cancer therapeutic strategies that possess both anti-cancer activity and capacity to target the cancer microenvironment to prevent cancer reoccurrence and/or metastasis. The anti-cancer strategies described herein rely on stimulating the activity of a potent anti-angiogenic and anti-tumorigenic protein, Thrombospondin 1 (Tsp-1). “Tsp-1” is a subunit of a disulfide-linked homotrimeric protein. Tsp-1 is an adhesive glycoprotein that mediates cell-to-cell and cell-to-matrix interactions. Tsp-1 binds to fibrinogen, fibronectin, laminin, type V collagen and integrins alpha-V/beta-1 and has been shown to play roles in platelet aggregation, angiogenesis, and tumorigenesis. For the purpose of the present disclosure, Tsp-1 is a potent anti-tumorigenic and anti-angiogenic factor, whose activation suppresses tumor growth and metastasis and represses angiogenesis in the tumor microenvironment.

As described herein, it has been discovered that tumor cells from several different types of cancers that are responsive Psap peptides present with dually elevated levels of CD36 and CD47.

Prosaposin or prosaposin-derived peptides were previously shown to be able to stimulate the activity of Tsp-1 and are effective for treating multiple types of cancers (see, e.g., PCT publications WO2009002931 WO/2011/084685 and WO/2013/096868, WO2015148801 and U.S. patent application Ser. Nos. 12/640,788 and 13/516,511, all of which are incorporated herein by reference in their entireties).

The progression of cancer to the metastatic stage is a major contributing factor to its lethality. In order for a tumor to form lethal metastases it must gain access to the vasculature or lymphatic system (intravasation), survive during transit, exit the vascular or lymphatic channels (extravasation), and proliferate at the metastatic site [1]. In this process, heterotypic signaling between the tumor and its microenvironment can affect tumor growth by regulating the production and secretion of factors that mediate tumor growth, angiogenesis, and the immune response. Two proteins, prosaposin and PRSS2, were identified through a functional proteomic screen, designed to identify secreted proteins that modulate Tsp-1 in the microenvironment [2]. Prosaposin is expressed preferentially by weakly metastatic tumors and stimulates Tsp-1 in the tumor microenvironment. Conversely, PRSS2 is preferentially expressed by highly metastatic cells and inhibits Tsp-1 expression in the tumor microenvironment. Tsp-1 inhibits tumor growth and progression via multi-modal activity, specifically: (1) It is a broadly acting anti-angiogenic factor, (2) It has direct anti-tumor activity against tumors that express CD36, and (3) It promotes macrophage phagocytosis and T-cell activation via binding to CD47 [3-5]. The Tsp-1 stimulating activity of prosaposin and the Tsp-1 repressing activity of PRSS2 have both been determined to be mediated via binding to LRP1. Provided herein are antibodies that mimic prosaposin's Tsp-1 stimulating activity and block the Tsp-1 repressing activity of PRSS2.

Prosaposin was first identified as a novel suppressor of tumor metastasis, and such inhibition was documented to be achieved by stimulating p53 and subsequently Tsp-1 in the tumor microenvironment [2]. A 5-amino acid cyclic peptide from prosaposin with potent anti-tumor and anti-metastatic activity has since been identified, referred to herein as VT1021. The peptide has been shown to inhibit metastasis by stimulating Tsp-1 in bone marrow-derived cells in the tumor microenvironment [6]. As such, the hypothesis that psap would have efficacy in treating metastatic pancreatic cancer, a cancer in which the microenvironment comprises the majority of the tumor mass, was tested [7]. Accordingly, 1×10⁶ AsPc1 human pancreatic cancer cells expressing firefly luciferase were injected into the pancreas of SCID mice. The tumors were allowed to grow for 25 days, at which point the luciferase intensity was greater than 1×10⁸ for all tumors. Treatment was then initiated with the psap peptide at doses of 20 mg/kg and 40 mg/kg QD. All mice were sacrificed after 21 days of treatment when the control (vehicle) treated mice became moribund.

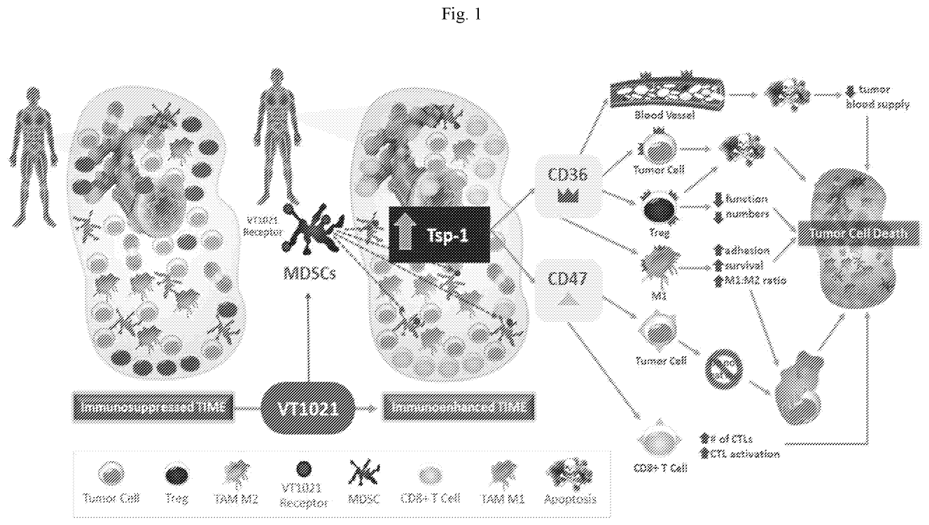

FIG. 1 illustrates VT1021 modulation of the tumor microenvironment (TME) via CD36 and CD47. VT1021 reprograms the TME from one that is immunosuppressive to one that is immunoenhanced. The immunosuppressed tumor immune microenvironment is characterized by high levels of Tregs, M2 tumor associated macrophages (M2 TAMs) and MDSCs. VT1021 binds to a receptor on MDSCsand induces expression of Tsp-1. Tsp-1 binding to one of its receptors, CD36, inhibits angiogenesis, induces apoptosis in tumor cells, endothelial cells and Tregs, and increases M1 macrophage adhesion, survival, and the M1: M2 ratio. Tsp-1 binding to its other major receptor, CD47, blocks the “do-not-eat-me” signal allowing macrophage phagocytosis of tumor cells and increases CTL infiltration and activity resulting in tumor cell death. Accordingly, VT1021 is an effective agent for simultaneous modulation of CD36 and CD47 for the treatment of cancer.

The present disclosure is based on the finding that dual high levels of CD36 and CD47 are predictive of the responsiveness of a subject to cancer treatment using Psap peptides, including CDWLPK (SEQ ID NO 1), DWLPK (SEQ ID NO 2), and DWLP (SEQ ID NO 3), amino acid substitution variant thereof, and cyclized versions thereof. Accordingly, aspects of the disclosure relate to methods for evaluating the responsiveness of a subject to treatment with a Psap peptide by determining a level of CD36 and CD47 in a sample, such as a tumor sample. In some embodiments, the methods described herein relate to identification or selection of a subject for treatment with a Psap peptide based on a level of CD36 and CD47 in a sample, such as a tumor sample. Other aspects of the disclosure relate to compositions and methods for treatment of a subject with cancer characterized by an elevated level of CD36 and CD47 (e.g., selected or identified on the basis that the cancer has an elevated level of CD36 and CD47 in a sample compared to a control level).

Provided herein are methods and compositions for the treatment of cancer by administering agents that modulate Tsp-1 activity. In some embodiments, the methods comprise determining the levels of CD36 and CD47 in a subject having cancer, and administering Tsp-1 inducing agents wherein the subject demonstrates cancer having elevated CD36 and CD47 levels or activity as compared to controls.

Aspects of the disclosure relate to performing an assay to determine levels of CD36 and CD47 in a sample. Any assay known in the art can be used for measuring CD36 and CD47 levels (see, e.g., Molecular Cloning: A Laboratory Manual, J. Sambrook, et al., eds., Third Edition, Cold Spring Harbor Laboratory Press, Cold Spring Harbor, New York, 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York). The levels of CD36 and CD47 can be mRNA levels and/or protein levels. In some embodiments, the levels are protein levels. Assays for detecting mRNA include, but are not limited to, Northern blot analysis, RT-PCR, sequencing technology, RNA in situ hybridization (using e.g., DNA or RNA probes to hybridize to RNA molecules present in the sample), in situ RT-PCR (e.g., as described in Nuovo G J, et al. Am J Surg Pathol. 1993, 17:683-90; Komminoth P, et al. Pathol Res Pract. 1994, 190:1017-25), and oligonucleotide microarray (e.g., by hybridization of polynucleotide sequences derived from a sample to oligonucleotides attached to a solid surface (e.g., a glass wafer) with addressable locations, such as an Affymetrix microarray (Affymetrix.RTM., Santa Clara, Calif.)). Methods for designing nucleic acid binding partners, such as probes, are well known in the art. In some embodiments, the nucleic acid binding partners bind to a part of or an entire nucleic acid sequence of CD36 or CD47, the sequence being identifiable with CD36 or CD47 known in the art.

Assays for detecting CD36 and CD47 protein levels include, but are not limited to, immunoassays (also referred to herein as immune-based or immuno-based assays, e.g., Western blot, immunohistochemistry and ELISA assays), Mass spectrometry, and multiplex bead-based assays. Such assays for protein level detection are well-known in the art. Binding partners for protein detection can be designed using methods known in the art and as described herein. In some embodiments, the CD36 and CD47 protein binding partners, e.g., anti-CD36 and CD47 antibodies, bind to a part of or an entire amino acid sequence of the CD36 and CD47 protein. Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

In some embodiments, the sample obtained from a subject is a tumor biopsy and the assay for detecting CD36 and CD47 protein levels is an immuno-based assay performed on the tumor biopsy.

In some embodiments, CD36 and CD47 are measured in a cancer cell or tumor.

In some embodiments, CD36 and CD47 are measured in tumor microenvironment, In some embodiments, CD36 and CD47 are measured in circulation.

Any suitable binding partner for CD36 or CD47 is contemplated for detection of a CD36 or CD47 level. In some embodiments, the binding partner is any molecule that binds specifically to CD36 or CD47 protein. As described herein, “binds specifically to CD36 or CD47 protein” means that the molecule is more likely to bind to a portion of or the entirety of CD36 or CD47 protein than to a portion of or the entirety of a non-CD36 or non-CD47 protein. In some embodiments, the binding partner is an antibody or antigen-binding fragment thereof, such as Fab, F (ab) 2, Fv, single chain antibodies, Fab and sFab fragments, F(ab′) 2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically to CD36 or CD47. Methods for producing peptide molecules and aptamers are well known in the art (see, e.g., published US Patent Application No. 2009/0075834, U.S. Pat. Nos. 7,435,542, 7,807,351, and 7,239,742).

In some embodiments, the binding partner is any molecule that binds specifically to CD36 or CD47 mRNA. As described herein, “binds specifically to CD36 or CD47 mRNA” means that the molecule is more likely to bind to a portion of or the entirety of the mRNA (e.g., by complementary base-pairing) than to a portion of or the entirety of a non-CD36 or non-CD47 mRNA nucleic acid. In some embodiments, the binding partner that binds specifically to a CD36 or CD47 mRNA is a nucleic acid, e.g., a probe. Binding partners can be designed using the nucleotide and amino acid sequences of CD36 or CD47, which are provided herein. In some embodiments, a CD36 or CD47 binding partner may comprise a detectable label, such as an enzymatically active group, a fluorescent molecule, a chromophore, a luminescent molecule, a specifically bindable ligand, or a radioisotope. In some embodiments, a second binding partner specific for the CD36 or CD47 binding partner is also contemplated, such as a secondary antibody.

Aspects of the disclosure relate to determining a level of CD36 or CD47 in a sample obtained from a subject. In some embodiments, the sample obtained from a subject is a tumor sample. As used herein, a tumor sample may comprise, e.g., a tumor cell, a population of tumor cells, a fragment of a tumor (e.g., a biopsy), or an entire tumor. In some embodiments, the tumor sample is a tumor biopsy. In some embodiments, the tumor sample comprises circulating tumor cells. In some embodiments, the tumor sample comprises ascites. In some embodiments, the tumor sample comprises pleural fluid. The tumor sample may contain non-tumor cells or non-tumor tissue (e.g., a biopsy that contains normal tissue surrounding a tumor fragment). In some embodiments, the sample may be a tissue or fluid sample obtained from a subject. Examples of fluid samples are blood, plasma, serum, and urine.

“Stimulate,” as used herein, means to activate or to increase the level or activity of a biological molecule (e.g., a protein). For example, the agent of the present disclosure “stimulates Tsp-1” means the expression level or activity level of Tsp1 is increased by at least 30% (e.g., at least 30%, at least 40%, at least 50%, at least 60%, at least 70%, at least 80%, at least 90%, at least 100%, at least 2-fold, at least 3-fold, at least 5-fold, at least 10-fold, at least 20-fold, at least 50-fold, at least 100-fold, or more) in the presence of the agent, as compared to without the agent.

“Inhibit,” as used herein, means to prevent expression, to reduce the level of a protein (e.g., CD36 or CD47), or to decrease the activity of a biological molecule (e.g., a protein). For example, an agent that inhibits the expression of CD36 or CD47 may prevent CD36 or CD47 from being expressed, or it may reduce the level of CD36 or CD47 by at least 30% (e.g., by at least 30%, at least 40%, at least 50%, at least 60%, at least 70%, at least 80%, at least 90%, at least 95%, at least 99%, or more), compared to in the absence of the agent.

In some embodiments, the Tsp-1 inducing agent is a Psap peptide. In some embodiments, the Psap peptide is a peptide comprising the sequence CDWLPK (SEQ ID NO 1), DWLPK (SEQ ID NO 2), or DWLP (SEQ ID NO 3), or an amino acid substitution variant thereof, wherein the amino acid substitution is:
a) Tyrosine (Y) for Tryptophan (W);
b) an amino acid substitution for Leucine (L) selected from Valine (V), Alanine (A) or Glycine (G), or a non-canonical amino acid of similar size, or a derivative thereof;
c) Arginine (R) for Lysine (K);
d) a D-isomer of Aspartic Acid (D) for an L-isomer of Aspartic Acid (D) and/or a D-isomer of Leucine (L) for a L-isomer of Leucine (L);
e) a D-isomer of Tryptophan (W) for an L-isomer of Tryptophan (W) and/or a D-isomer of Proline (P) for an L-isomer of Proline (P); or combinations thereof. In some embodiments, the Psap peptide is 50 amino acids or fewer in length. In some embodiments, the Psap peptide is 30 amino acids or fewer in length. In some embodiments, the Psap peptide is 15 amino acids or fewer in length. In some embodiments, the Psap peptide is 6 amino acids or fewer in length. In some embodiments, the Psap peptide is a cyclic peptide. In some embodiments, the non-canonical amino acid of similar size is methylvaline, methylleucine, or sarcosine. In some embodiments, the peptide is cyclic DWLPK (SEQ ID NO. 2). As used herein, “VT1021” refers to cyclic DWLPK (SEQ ID NO. 2).

In some embodiments, the Psap peptide may be modified, for example, through oligomerization or polymerization (e.g., dimers, trimer, multimers, etc.), modifications of amino acid residues or peptide backbone, cross-linking, cyclization, conjugation, pegylation, glycosylation, acetylation, phosphorylation, fusion to additional heterologous amino acid sequences (for example, an antibody or antibody Fc domain, serum transferrin or portions thereof, albumin, or transthyretin), or other modifications that substantially alter the stability, solubility, or other properties of the peptide while substantially retaining or enhancing therapeutic activity. Conjugation may be, e.g., to a polymer. Suitable polymers include, for example, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and .alpha.,.beta.-Poly [(2-hydroxyethyl)-DL-aspartamide, and the like, or mixtures thereof. Conjugation may be through a linker, e.g., a peptide or chemical linker. Methods of modifying peptides are well known in the art (see, e.g., U.S. Pat. Nos. 5,180,816, 5,596,078, 5,990,273, 5,766,897, 5,856,456, 6,423,685, 6,884,780, 7,610,156, 7,256,258, 7,589,170 and 7,022,673, and PCT publication WO 2010/014616, the contents of which are incorporated herein by reference).

In some embodiments, the Tsp-1 inducing agent comprises a mixture of Tsp-1 inducing agents disclosed herein. As used herein, “Tsp-1 inducing agent” refers to an agent that promotes an increase in Tsp-1 levels or activity. In some embodiments, promoting Tsp-1 activity comprises increasing Tsp-1 expression, or the half-life of Tsp-1 protein. In some embodiments, promoting Tsp-1 activity comprises restoring a wild-type level of Tsp-1 levels or activity. In some embodiments, promoting Tsp-1 activity comprises increasing Tsp-1 levels or activity to more than wild-type levels. In some embodiments, promoting Tsp-1 levels or activity comprises increasing Tsp-1 levels or activity that remain lower than wild-type but that are increase as compared to a control.

The term “bind” refers to the association of two entities (e.g., two proteins). Two entities (e.g., two proteins) are considered to bind to each other when the affinity (KD) between them is <10⁻⁴ M, <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M. One skilled in the art is familiar with how to assess the affinity of two entities (e.g., two proteins).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

A peptide that is “derived from” a protein means the peptide is obtained from the protein and has an amino acid sequence that shares homology with the fragment of the protein it corresponds to. The amino acid sequence of the peptide may be at least 70%, at least 75%, at least 80%, at least 85%, at least 90%, at least 95%, at least 99% or 100% identical to the amino acid sequence of the fragment of the protein it corresponds to. A peptide that is derived from a protein may also contain chemical modifications, amino acid substitutions, and/or unnatural amino acids.

An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antigen binding fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervaricapacity, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are predicted to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and a classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the varicapacity is not evenly distributed across the 110-amino acid span of the variable domains. Instead, the V regions consist of relatively invariant stretches called framework regions (FRs) of 15-30 amino acids separated by shorter regions of extreme varicapacity called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

An “antigen binding fragment” for use in accordance with the present disclosure contains the antigen-binding portion of an antibody. The antigen-binding portion of an antibody refers to one or more fragments of an antibody that retain the capacity to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antigen binding fragments are obtained using conventional techniques known to those with skill in the art, and the fragments are screened for utility in the same manner as are full-length antibodies.

In some embodiments, an antigen binding fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antigen binding fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the capacity to recognize and bind antigen, although at a lower affinity than the entire binding site.

Single-chain Fv also abbreviated as “sFv” or “scFv” are antigen binding fragments that comprise the VH and VL antibody domains connected into a single polypeptide chain. Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

Antibodies may be isolated. An isolated antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In some embodiments, the antibody of the present disclosure is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present technology may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibody of the present technology is a polyclonal antibody. A “polyclonal antibody” a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

Methods of producing antibodies (e.g., monoclonal antibodies or polyclonal antibodies) are known in the art. For example, a polyclonal antibody may be prepared by immunizing an animal, preferably a mammal, with an allergen of choice followed by the isolation of antibody-producing B-lymphocytes from blood, bone marrow, lymph nodes, or spleen. Alternatively, antibody-producing cells may be isolated from an animal and exposed to an allergen in vitro against which antibodies are to be raised. The antibody-producing cells may then be cultured to obtain a population of antibody-producing cells, optionally after fusion to an immortalized cell line such as a myeloma. In some embodiments, as a starting material B-lymphocytes may be isolated from the tissue of an allergic patient, in order to generate fully human polyclonal antibodies. Antibodies may be produced in mice, rats, pigs (swine), sheep, bovine material, or other animals transgenic for the human immunoglobulin genes, as starting material in order to generate fully human polyclonal antibodies. In some embodiments, mice or other animals transgenic for the human immunoglobulin genes (e.g. as disclosed in U.S. Pat. No. 5,939,598), the animals may be immunized to stimulate the in vivo generation of specific antibodies and antibody producing cells before preparation of the polyclonal antibodies from the animal by extraction of B lymphocytes or purification of polyclonal serum.

Monoclonal antibodies are typically made by cell culture that involves fusing myeloma cells with mouse spleen cells immunized with the desired antigen (i.e., hyrbidoma technology). The mixture of cells is diluted and clones are grown from single parent cells on microtitre wells. The antibodies secreted by the different clones are then assayed for their capacity to bind to the antigen (with a test such as ELISA or Antigen Microarray Assay) or immuno-dot blot. The most productive and stable clone is then selected for future use.

In some embodiments, the antibodies described herein are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and cap capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “small molecule,” as used herein, refers to a molecule of low molecular weight (e.g., <900 daltons) organic or inorganic compound that may function in regulating a biological process. Nonlimiting examples of a small molecule include lipids, monosaccharides, second messengers, other natural products and metabolites, as well as drugs and other xenobiotics.

A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relay signals received at receptors on the cell surface (e.g., from protein hormones, growth factors, etc.) to target molecules in the cytosol and/or nucleus. Nonlimiting examples of second messenger molecules include cyclic AMP, cyclic GMP, inositol trisphosphate, diacylglycerol, and calcium. A “metabolite” is an molecule that forms as an intermediate produce of metabolism. Non-limiting examples of a metabolite include ethanol, glutamic acid, aspartic acid, 5′ guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs, antibiotics, carcinogens, environmental pollutants, food additives, hydrocarbons, and pesticides.

Therapeutic agents described herein may be formulated in a pharmaceutical composition. In some embodiments, the pharmaceutical composition further comprises a pharmaceutical acceptable carrier. The term “pharmaceutically-acceptable carrier” as used herein means one or more compatible solid or liquid filler, diluents or encapsulating substances which are suitable for administration into a subject, e.g., a human. A pharmaceutically acceptable carrier is “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

Other aspects of the present disclosure provide methods of treating cancer, using the agents and pharmaceutical compositions described herein. In some embodiments, the method comprises administering to a subject in need thereof an effective amount of an agent that stimulates Tsp-1. In some embodiments, the method comprises administering to the subject in need thereof one or more additional agents for inducing Tsp-1 or suppressing CD36 or CD47. When more than one agent is administered, they may be administered simultaneously or sequentially. One skilled in the art (e.g., a physician) is able to determine the mode of administration.

“Treat” or “treatment” of cancer includes, but is not limited to, preventing, reducing, or halting the development of a cancer, reducing or eliminating the symptoms of cancer, suppressing or inhibiting the growth of a cancer, preventing or reducing metastasis and/or invasion of an existing cancer, promoting or inducing regression of the cancer, inhibiting or suppressing the proliferation of cancerous cells, reducing angiogenesis and/or increasing the amount of apoptotic cancer cells.

An “effective amount” is a dosage of an agent sufficient to provide a medically desirable result, such as treatment of cancer. The effective amount will vary with the particular disease or disorder being treated, the age and physical condition of the subject being treated, the severity of the condition, the duration of the treatment, the nature of any concurrent therapy, the specific route of administration and the like factors within the knowledge and expertise of the health practitioner. For administration to a subject such as a human, a dosage of from about 0.001, 0.01, 0.1, or 1 mg/kg up to 50, 100, 150, or 500 mg/kg or more can typically be employed.

In some embodiments, the method further comprises identifying the subject with elevated levels of CD36 and CD47 in the sample compared to the control level as responsive to or likely to be responsive to treatment with a Psap peptide. In some embodiments, the method further comprises administering to the subject identified as responsive to or likely to be responsive to treatment with a Psap peptide an effective amount of a Psap peptide described herein to treat the cancer. In some embodiments, the sample obtained from a subject having cancer is a tumor sample.

In some embodiments, elevated levels of CD36 and CD47 in the sample compared to a control level indicates that the cancer will regress or is likely to regress in response to treatment with a Psap peptide. In some embodiments, the method further comprises identifying the subject with elevated levels of CD36 and CD47 in the sample compared to the control level as having a cancer that will regress or is likely to regress in response to treatment with a Psap peptide. In some embodiments, the method further comprises administering to the subject identified having a cancer that will regress or is likely to regress in response to treatment with a Psap peptide an effective amount of a Psap peptide described herein to cause regression of the cancer.

As used herein, “elevated levels of CD36 and CD47” means that the levels of CD36D47 are above a control level, such as a pre-determined threshold or a level, levels measured in a non-cancerous sample from the same subject, or levels measured in a sample from a healthy subject or population of subjects.

Control levels are described in detail herein. Elevated levels of CD36 and CD47 include levels that are, for example, 1%, 5%, 10%, 20%, 30%, 40%, 50%, 60%, 70%, 80%, 90%, 100%, 150%, 200%, 300%, 400%, 500% or more above a control level. Elevated levels of CD36 and CD47 also includes increasing a phenomenon from a zero state (e.g., no or undetectable expression in a control) to a non-zero state (e.g., some expression or detectable expression in a sample).

As used herein, “treatment with a Psap peptide” is meant to comprise administration of a Psap peptide to a subject. Psap peptides are described herein. It is to be understood that treatment with a Psap peptide may include treatment with only a Psap peptide or may include treatment with multiple agents or therapies, such as a Psap peptide and another chemotherapeutic agent and/or another form of therapy such as surgery, radiotherapy, or chemotherapy.

As used herein, “responsive to treatment with a Psap peptide” includes, but is not limited to, prevention or reduction of the development of a cancer, reduction of the symptoms of cancer, suppression or inhibition of the growth of a cancer, prevention of metastasis and/or invasion of an existing cancer, promotion or induction of regression of the cancer, inhibition or suppression of the proliferation of cancerous cells, reduction of angiogenesis and/or an increase in the amount of apoptotic cancer cells in response to treatment with a Psap peptide.

As used herein, “non-responsive to treatment with a Psap peptide” includes, but is not limited to, an absence of prevention or reduction of the development of a cancer, an absence of reduction of the symptoms of cancer, an absence of suppression or inhibition of the growth of a cancer, an absence of prevention of metastasis and/or invasion of an existing cancer, an absence of promotion or induction of regression of the cancer, an absence of inhibition or suppression of the proliferation of cancerous cells, an absence of reduction of angiogenesis and/or a decrease in the amount of apoptotic cancer cells in response to treatment with a Psap peptide.

In some embodiments, the effective amount is a dosage of an agent that causes no toxicity to the subject. In some embodiments, the effective amount is a dosage of an agent that causes reduced toxicity to the subject. Methods for measuring toxicity are well known in the art (e.g., biopsy/histology of the liver, spleen, and/or kidney; alanine transferase, alkaline phosphatase and bilirubin assays for liver toxicity; and creatinine levels for kidney toxicity).

The agents and pharmaceutical compositions described herein can be formulated for a variety of modes of administration, including systemic, topical or localized administration. A variety of administration routes are available. The particular mode selected will depend upon the type of cancer being treated and the dosage required for therapeutic efficacy. The methods of the disclosure, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, or parenteral routes. The term “parenteral” includes subcutaneous, intravenous, intramuscular, or infusion. The pharmaceutical compositions described herein are also suitably administered by intratumoral, peritumoral, intralesional, intratracheal, intracerebroventricular, intraperitoneal or perilesional routes, to exert local as well as systemic effects.

Techniques and formulations generally can be found in Remington: The Science and Practice of Pharmacy, Pharmaceutical Press; 22nd edition and other similar references. When administered, a Psap peptide may be applied in pharmaceutically-acceptable amounts and in pharmaceutically-acceptable compositions. Pharmaceutical compositions and pharmaceutically-acceptable carriers are also described herein. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the disclosure. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

In some embodiments, treatment of cancer with the agents or pharmaceutical compositions described may be combined with another therapy, such as a chemotherapy agent, radiation, a cytostatic agent, an anti-VEGF agent, an anti-angiogenesis factor, a p53 reactivation agent and/or surgery.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof. A subject in need thereof can be a subject who has a risk of developing cancer (i.e., via a genetic test) or a subject who has cancer.

Subjects having cancer may be identified using any method known in the art (e.g., blood tests, histology, CT scan, X-ray, MRI, physical exam, cytogenitic analysis, urinalysis, or genetic testing). A subject suspected of having cancer might show one or more symptoms of the disease. Signs and symptoms for cancer are well known to those of ordinary skill in the art. Some exemplary laboratory tests include, but are not limited to, testing for cancer biomarkers such as cancer antigen (CA) 15-3, carcinoembryonic antigen (CEA) and HER-2 for breast cancer, human papillomavirus (HPV) E6 and E7 oncoproteins for cervical cancer, alpha-fetoprotein (AFP), AFP fractions L3, P4/5, and the +II band, and ultrasonography for hepatocellular carcinoma (HCC), prostate-specific antigen (PSA) for prostate cancer, and serum CA-125 for ovarian and HCC.

The cancer can be benign or malignant, and it may or may not have metastasized. Any type of cancer is contemplated herein, including, but not limited to, leukemias, lymphomas, myelomas, carcinomas, metastatic carcinomas, sarcomas, adenomas, nervous system cancers and genitourinary cancers. Exemplary cancer types include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is melanoma or ovarian cancer.

### Examples

### Example 1: Dually Elevated Levels of CD36 and CD47 Correlate with Responsiveness to VT1021 Treatment in Subjects with Pancreatic Cancer

This example demonstrates that dually elevated levels of CD36 and CD47 correlated with responsivity to VT2021 treatment in pancreatic cancer.

Preclinical studies of VT1021 have shown robust anti-tumor activity in multiple animal models of ovarian, pancreatic, and breast cancer. VT1021 was evaluated in a recently completed phase I/II open-label, multicenter, dose escalation (Part 1) and expansion (Part 2) clinical study in advanced, refractory, solid tumors including pancreatic cancer (NCT03364400). Seven subjects with pancreatic cancer were dosed in Part 1 and 32 were dosed in Part 2, 17 of which were considered evaluable as having completed ≥1 cycle of VT1021 treatment and tumor imaging during cycle 2. VT1021 had no major adverse events (AEs) and a predictable pharmacokinetic profile.

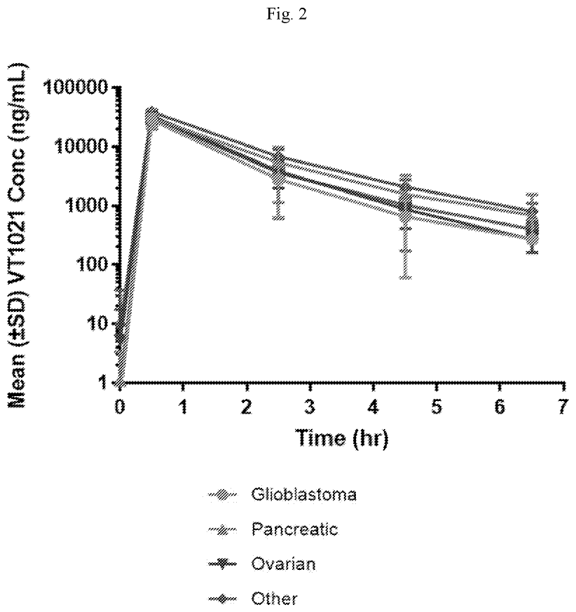

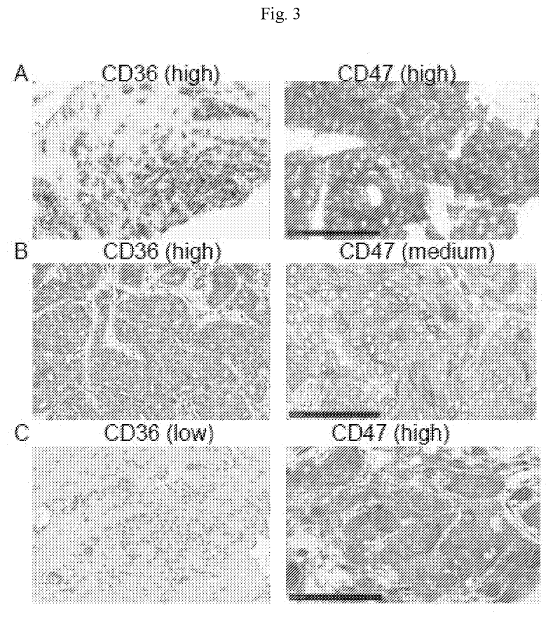

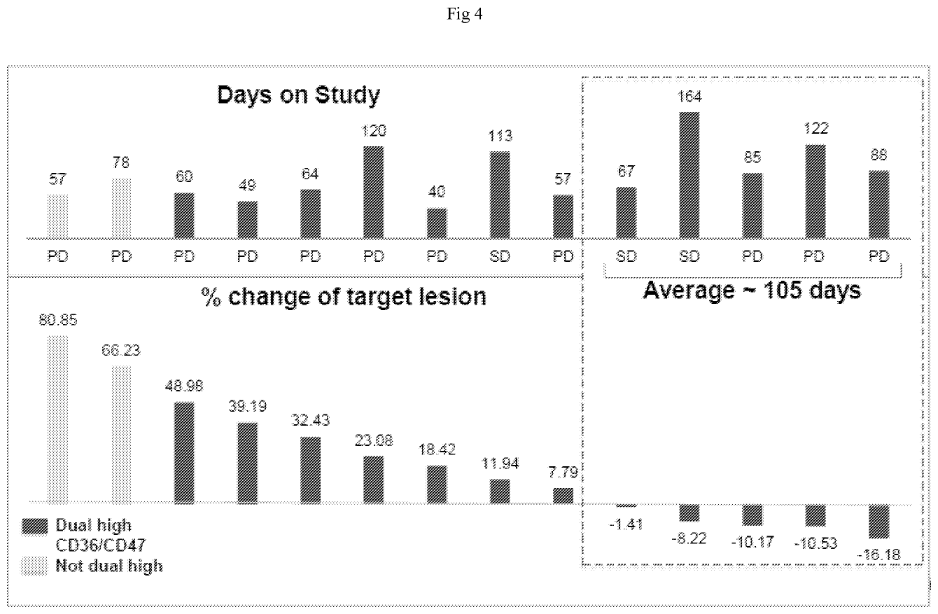

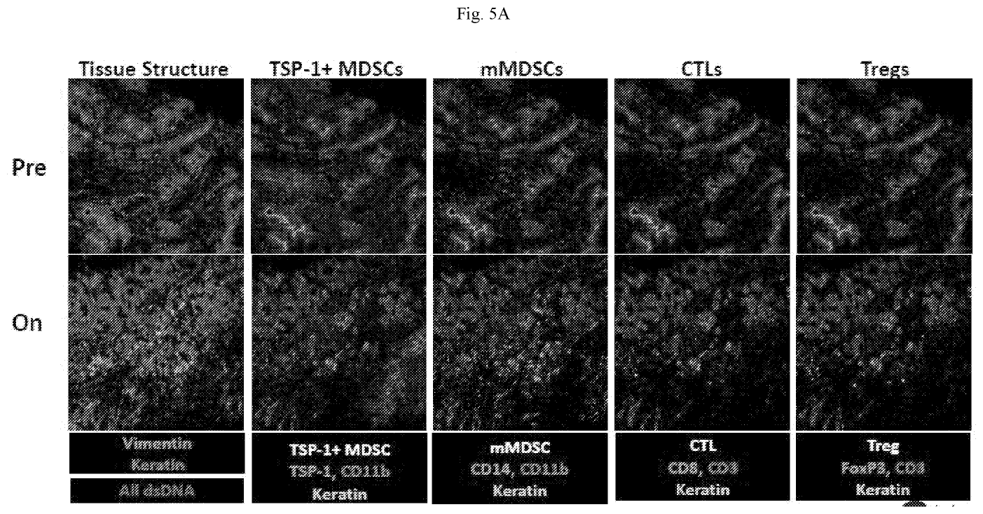

In the “expansion stage” of the VT1021-01 Study, subjects diagnosed with recurrent GBM, pancreatic cancer, ovarian cancer and other solid tumors were enrolled. They were treated with VT1021 at 11.8 mg/kg, twice weekly by IV infusion. The blood samples were collected from the clinical study at pre-treatment, 0, 2, 4, 6 hours post treatment to measure the levels of VT1021 (FIG. 2). The tumor biopsy samples prior to treatment and during treatment were collected from the clinical study to measure expression levels of biomarkers including CD36 and CD47, as well as cell surface markers for immune cell populations in the TME by immunohistochemistry assays (FIG. 3, FIG. 4, FIG. 5). The correlation between days on study and tumor shrinkage versus expression profile of CD36 and CD47 was analyzed (FIG. 4). The general expression profile of CD36 and CD47 has been assessed by IHC on Tumor Tissue Microarrays (TMAs), which contain biopsy samples collected from pancreatic cancer patients. TMAs were purchased from commercial sources, not related to the VT1021-01 clinical study.

Common adverse effects (AEs) in escalation subjects with pancreatic cancer (Part 1) are shown in Table 1 The AEs shown occurred in 2 or more subjects, all cycles, maximum grade, regardless of toxicity).

Of 39 pancreatic cancer subjects enrolled and dosed, 7 subjects were enrolled in the Part 1 escalation cohort. All 7 subjects reported at least one AE (regardless of causality). Four subjects had at least one AE, which was probably or possibly related to the study drug. There was no dose-limiting toxicity. Five subjects reported at least one severe adverse event (SAE) (regardless of causality). Two subjects had SAEs definitely or possibly related to study drug: infusion related reaction (grade 3), altered mental status (grade 2).

Thirty-two subjects were enrolled in the Part 2 escalation cohort. Thirty subjects reported at least one AE (regardless of causality). Eighteen subjects had at least one AE definitely, probably or possibly related to the study drug. Twenty subjects reported at least one SAE (regardless of causality). Two subjects had SAEs possibly related to study drug: thrombosis (grade 3), fever (grade 1)

TABLE 1
Common AEs in escalation (Part 1) subjects with pancreatic cancer (AEs that
occurred in ≥2 subjects, all cycles, maximum grade, regardless of toxicity)

| AE Term | Total | Grade 1 | Grade 2 | Grade 3 |
|---|---|---|---|---|
| Abdominal pain | 4 | 3 | | 1 |
| Constipation | 2 | 1 | | 1 |
| Nausea | 2 | 2 | | |
| Fatigue | 2 | 2 | | |
| Urinary tract infection | 2 | | 2 | |
| Infusion related reaction | 2 | | 1 | 1 |
| Thrombosis | 2 | | | 2 |

Common AEs in expansion (Part 2) subjects with pancreatic cancer (AEs that
occurred in ≥6 subjects, all cycles, maximum grade, regardless of toxicity)

| AE Term | Total | Grade 1 | Grade 2 | Grade 3 |
|---|---|---|---|---|
| Anaemia | 7 | 4 | 2 | 1 |
| Abdominal pain | 16 | 3 | 9 | 4 |
| Constipation | 8 | 4 | 4 | |
| Diarrhoea | 8 | 5 | 3 | |
| Nausea | 14 | 10 | 3 | 1 |
| Fatigue | 14 | 3 | 8 | 3 |
| Pyrexia | 6 | 6 | | |
| Alanine aminotransferase increased | 6 | 4 | | 2 |
| Aspartate aminotransferase increased | 6 | 2 | 2 | 2 |
| Blood bilirubin increased | 7 | | 3 | 4 |
| Decreased appetite | 11 | 7 | 2 | 2 |
| Insomnia | 6 | 6 | | |

FIG. 2 is a chart showing VT1021 pharmacokinetics in subjects having glioblastoma, pancreatic cancer, ovarian cancer.

FIG. 3 shows the expression intensities of CD36 and CD47 in subjects with pancreatic cancer. Dual high expression of both CD36 and CD47 in an evaluable subject is shown in part A. Nondual high expression of CD36 and CD47 in an evaluable subject is shown in part B. Nondual high expression of CD36 and CD47 in a non-evaluable subject is shown in part C.

FIG. 4 demonstrated that dual high expression of CD36 and CD47 correlates with longer days on the study and reduced tumor burden. A waterfall plot illustrates changes in tumor burden for 14 subjects in Part 2 with pancreatic cancer with measurable disease. Three out of 3 subjects with stable disease (100%) were showed dually high CD36 and CD47 levels. Of the 14 subjects with measurable disease, all 5 subjects with reduction of tumor burden showed dually high levels and remained on the study for an average of 105 days.

FIG. 5A and FIG. 5 B are charts showing that VT1021 modulates the TME in pancreatic cancer. Modulation of the TME by VT1021 in paired biopsies from subjects with pancreatic cancer was assessed by multiplex ion beam image analysis. Representative images from 1 of 3 regions of interest in tumor biopsies from a subject obtained pre-treatment (pre) and on-study (on). FIG. 5B shows the TSP-1+mean intensity, the ratio of CTLs to Tregs, the ratio of M1 to M2 macrophages, and the fold change of percent of M1 macrophages compared to total macrophages were quantified for 8 pairs of biopsies from Parts 1 & 2. TSP-1+increased >2 fold in on-study biopsies compared to pre-treatment, both the CTL/Treg and M1/M2 macrophage ratios increased and the percent of M1 macrophages increased >3 fold in the TME after treatment with VT1021.

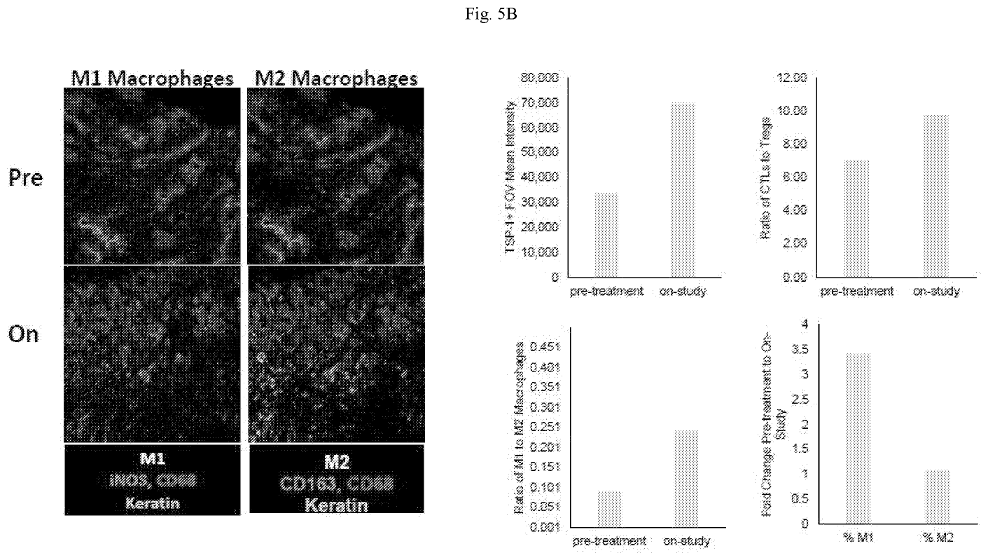

FIG. 6 shows that dual high expression of CD36 and CD47 is a predictive biomarker for pancreatic cancer. A high percentage of dually high CD36 and CD47 is observed in pancreatic cancer. The figure shows representative images of commercially available pancreatic tumor tissue microarrays stained and scored for dual high CD36 and CD47. Table 2, below, shows that Part 2 subjects with dually high CD36 and CD47 were more likely to be considered evaluable in the Phase 1 study with VT1021.

TABLE 2

| Pancreatic Cancer | Cases | Dual High |
|---|---|---|
| All enrolled (Part 2) | 32 | 63% |
| Evaluable (Part 2) | 17 | 88% |
| TMA | 100 | 56% |
| TMA-Stage IV | 22 | 53% |

These data show that pancreatic cancer subjects presenting with dually high CD36 and CD47 levels are more likely to have a reduction in tumor burden and remain on clinical studies longer than subjects not presenting with dually high CD36 and CD47 levels. Increased TSP-1 expression is observed in the TME in subjects receiving VT1021, and VT1021 remodels the TME to be more immune sensitive via increased CTL/Tregs and M1 macrophage accumulation. Accordingly, dually high CD36 and CD47 levels is a predictive biomarker for the responsiveness of pancreatic cancer subjects to treatment with VT1021.

These results show that dually elevated levels of CD36 and CD47 is a robust biomarker for the responsivity of a cancer to treatment with VT1021, including the responsivity of pancreatic cancer. The results further show that peptides of the present technology, including VT1021, are useful for the treatment of cancers presenting with dually elevated levels of CD36 and CD47 as compared to control levels, including pancreatic cancer.

### Example 2: Dually Elevated Levels of CD36 and CD47 Correlate with Responsiveness to VT1021 Treatment in Subjects with Glioblastoma

This example demonstrates that dually elevated levels of CD36 and CD47 correlated with responsivity to VT2021 treatment in glioblastoma.

Preclinical studies of VT1021 have shown robust anti-tumor activity in multiple animal models of ovarian, pancreatic, and breast cancer. VT1021 was evaluated in a recently completed phase I/II open-label, multicenter, dose escalation (Part 1) and expansion (Part 2) clinical study in advanced, refractory, solid tumors including recurrent GBM (rGBM) (NCT03364400). Thirty-two subjects with rGBM were dosed in Part 2, 22 of which were considered evaluable as having completed ≥1 cycle of VT1021 treatment and tumor imaging during cycle 2. VT1021 had no major adverse events (AEs) and a predictable pharmacokinetic profile. Table 3, below, shows subject demographics. Table 4 shows the incidence of adverse effects.

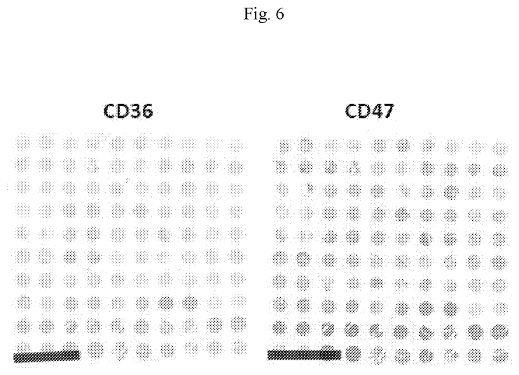

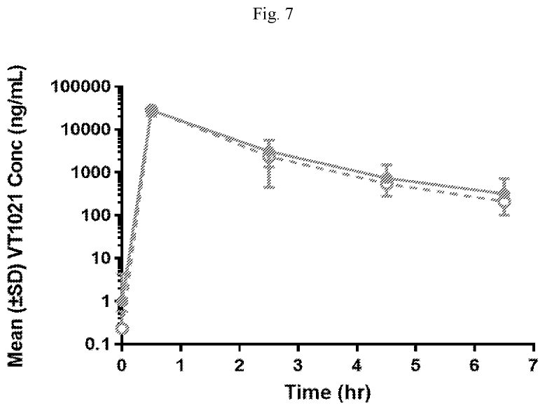

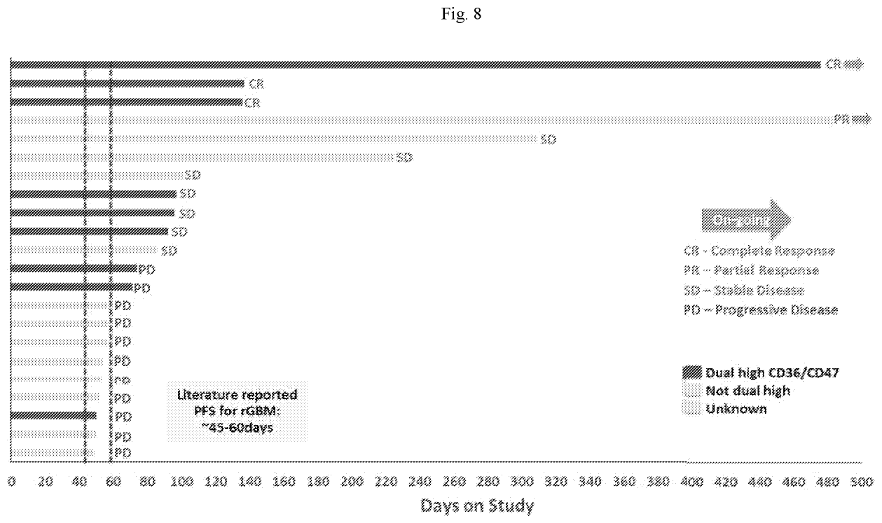

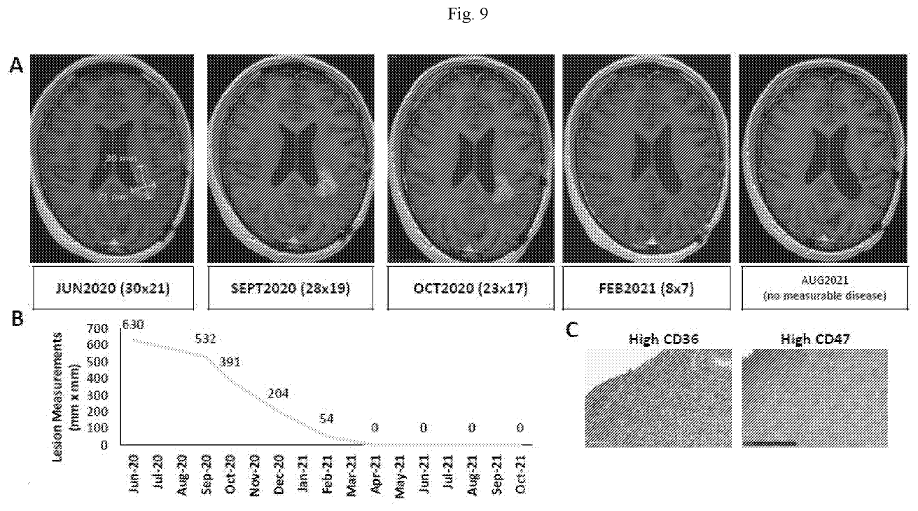

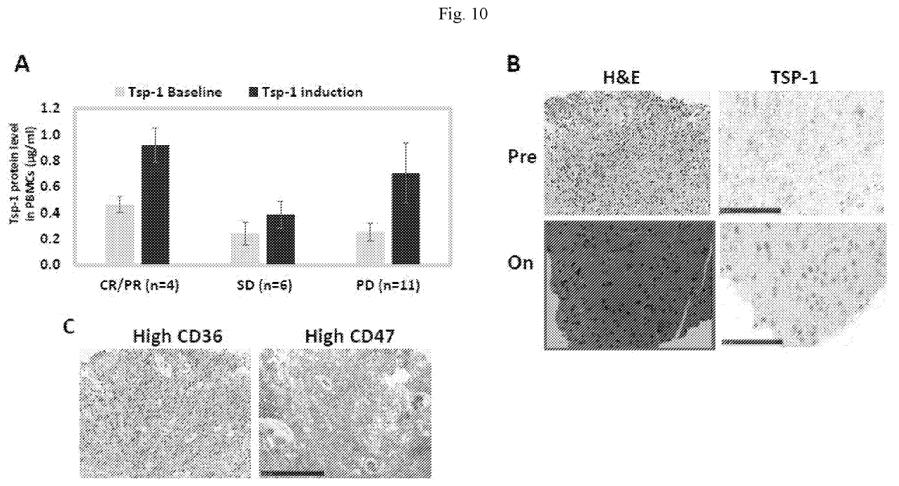

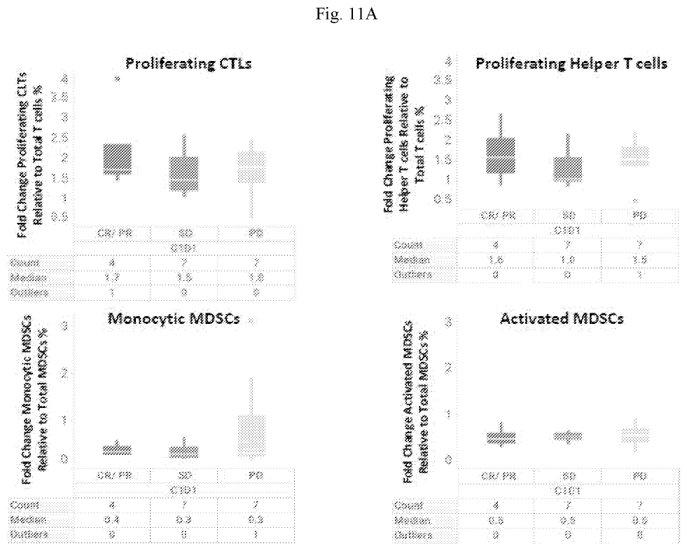

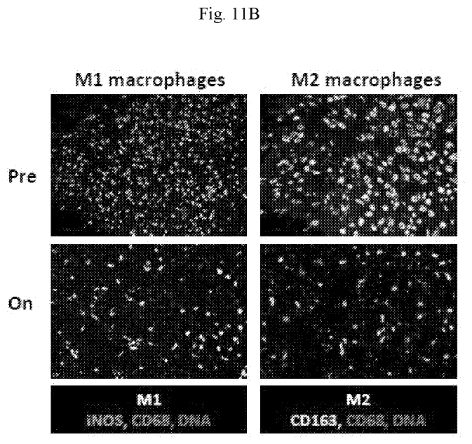

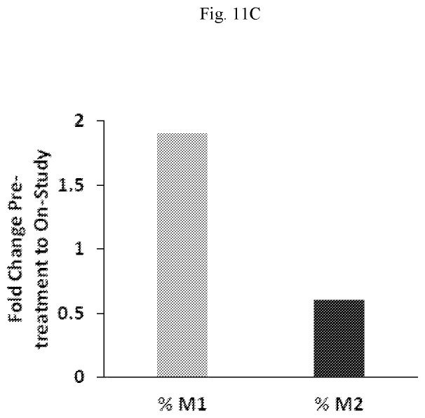

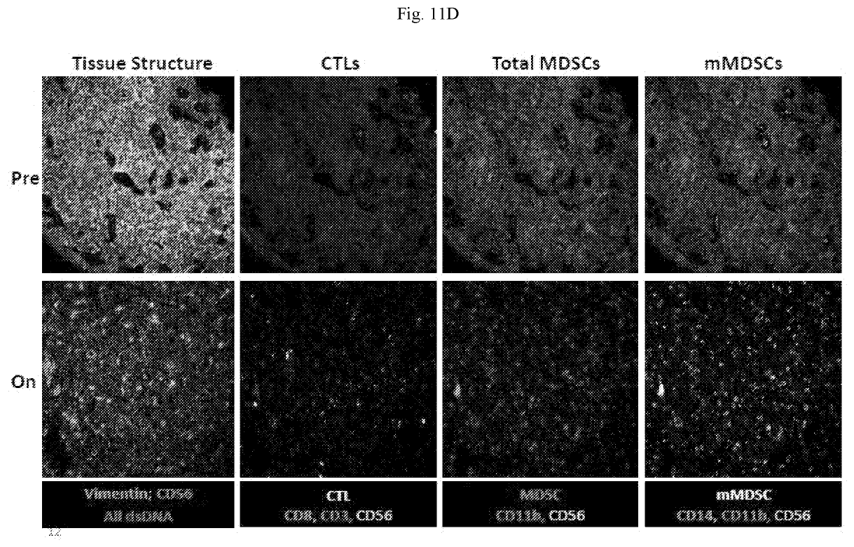

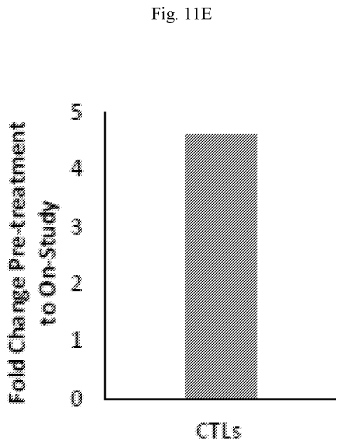

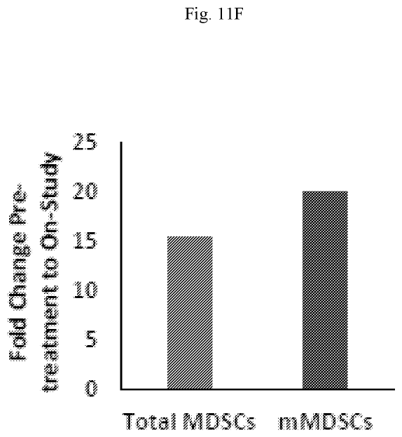

The blood samples were collected from GBM subjects in the expansion phase, who were treated by VT1021 at 11.8 mg/kg, twice weekly, by IV infusion. VT1021 levels were measured and analyzed among male and female subjects (FIG. 7). Days on study have been collected from the same group of subjects (FIG. 8). One subject achieved “complete response” as demonstrated by MRI scans and measurement of lesion sizes over the course of treatment by VT1021 (FIG. 9). CD36 and CD47 levels have been measured by immunohistochemistry assays in the pre-treatment tumor biopsy samples of the GBM subjects (FIG. 9, FIG. 10). Levels of Tsp-1 were measured by ELISA for circulating Tsp-1 levels in the collected blood samples, and by immunohistochemistry for the level of Tsp-1 accumulated in the tumor biopsy samples (FIG. 10). The infiltration of immune and inflammatory cells, specifically, MDSCs, T cells and macrophages, were determined by immunohistochemical analysis of the levels of cell surface markers in the tumor biopsy samples of subjects (FIG. 11).

TABLE 3

| | | Statistics | Total (N = 32) |
|---|---|---|---|
| Age (years) | | Mean (STD) | 56.6 (14.8) |
| | | Median (Min, Max) | 59.5 (24, 82) |
| Age Group: | 18 <= to <= 65 | n (%) | 21 (65.6%) |
| | 66 <= to <= 75 | n (%) | 9 (28.1%) |
| | >75 | n (%) | 2 (6.3%) |
| Gender: | Female | n (%) | 16 (50%) |
| | Male | n (%) | 16 (50%) |

TABLE 4
Common Adverse Events: 32 Subjects
(All cycles, maximum grade, regardless of causality)

| | All Grades | Grade 1-2 | Grade 3-4 |
|---|---|---|---|
| Abdominal Pain | 5 | 5 | — |
| Diarrhea | 4 | 4 | — |
| Nausea | 3 | 3 | — |
| Vomiting | 3 | 2 | 1 |
| Fall | 9 | 9 | — |
| Infusion reaction | 3 | 3 | — |

There were 373 AE events reported in all GBM subjects treated (N = 32), of which 35 were SAEs
NO SAE was considered by site PIs and by sponsor monitor to be related to study drug

FIG. 7 is a chart showing VT1021 pharmacokinetics in subjects having glioblastoma.

FIG. 8 shows that dually high expression of CD36 and CD47 correlate with clinical response and duration of treatment. Among 22 evaluable rGBM subjects, 3 had complete response (CR), 1 had partial response (PR), and 7 had stable disease (SD) with an average study duration of over 203 days. The overall disease control rate (DCR) was 50%. Nine of the 20 (45%) evaluable subjects with available biopsy samples showed high expression levels of both CD36 and CD47. Among the 9 dual-high subjects, 3 achieved CR, representing an overall response rate of 33.3%, with another 3 subjects achieving SD for a DCR of 67%. Of the 11 CR/PR/SDs, 6 were dual high (55%).

FIG. 9 shows that VT1021 induces a complete response in rGBM subjects. Complete response scan images and lesion shrinkage for 1 of 3 subjects who achieved CR. Part A shows lesion measurements and scan images for 1 CR subject who has been on study for 476 days and is still on-going. Part B shows that the lesion steadily decreased until no longer measurable after 9 cycles of treatment. Part C shows dual high expression of CD36 and CD47 by IHC analysis of the pretreatment biopsy.

FIG. 10 shows that VT1021 induces TSP-1 in the circulation and the TME. Part A shows TSP-1 protein measured from peripheral blood mononuclear cells (PBMCs) was induced by VT1021 in all evaluable subjects with rGBM. Part B shows TSP-1 induction in the on-study (on) biopsy from a subject with rGBM who achieved CR. No tumor cells were detected by pathological examination of the on-study biopsy. Part C shows dual high expression intensities of CD36 and CD47 in a pre-treatment (pre) biopsy from the same subject of Part B.

FIG. 11A, FIG. 11B, FIG. 11C, FIG. 11D, FIG. 11E, and FIG. 11F are charts showing modulation of the immune system by VT1021 in circulation and in the TME. FIG. 11A shows immune cells isolated from whole blood collected before and after 6 hours of treatment with VT1021 on Cycle 1 Day 1 were analyzed by flow cytometry. Proliferating CTLs increased ≥1.5 fold regardless of clinical response, proliferating helper T cells increased ≥1.5 fold in CR/PR and PD subjects while monocytic MDSCs (mMDSCs) and activated MDSCs decreased≤0.5 fold in all evaluable subjects. FIG. 11B shows IHC for M1 and M2 macrophages on tumor biopsies obtained pre-treatment (pre) and on-study (on) from a subject with rGBM who achieved CR. FIG. 11C shows fold change of M1 and M2 macrophages were quantified for the biopsy pair in FIG. 11B. Percent of M1 increased 1.9-fold while M2 decreased 0.6-fold after treatment. FIG. 11D shows representative in tumor biopsies obtained pre-treatment and on-study from the subject in FIG. 11B. assessed by multiplex ion beam imaging. FIG. 11E shows fold change of CTLs. FIG. 11F shows total and mMDSCs were quantified for the biopsy pair in FIG. 11D. CTLs increased >4 fold while total and mMDSCs increased >15 fold in the on-study biopsy compared to pre-treatment.

These data show that VT1021 is an effective as a single-agent clinical activity in rGBM, particularly in subjects with high expression levels of CD36 and CD47. Subjects with rGBM who presented with dually high CD36 and CD47 levels were more likely to have a reduction in tumor burden and to stay in a clinical study longer than subjects not presenting with dually high CD36 and CD47 levels. Increased TSP-1 expression was observed in circulating PBMCs and in the TME, and VT1021 remodeled the TME to be more immune enhanced via increased M1 macrophages and CTLs. Dually high expression of CD36 and CD47 is a accordingly a predictive biomarker for the responsiveness of GBM subjects to treatment with VT1021.

These results show that dually elevated levels of CD36 and CD47 is a robust biomarker for the responsivity of a cancer to treatment with VT1021, including the responsivity of glioblastoma. The results further show that peptides of the present technology, including VT1021, are useful for the treatment of cancers presenting with dually elevated levels of CD36 and CD47 as compared to control levels, including glioblastoma.

### Example 3: Treatment of Pancreatic Cancer Having Dually Elevated Levels of CD36 and CD47 Using VT1021

This example will demonstrate the efficacy of VT1021 for the treatment of pancreatic cancers presenting with dually elevated levels of CD36 and CD47.

Subjects having pancreatic cancer are identified using standard clinical protocols. Samples are obtained and levels of CD36 and CD47 measured using methods known in the art. Subjects presenting with dually elevated CD36 and CD47 levels as compared to suitable controls are selected for dosing with VT1021.

Subjects advancing to the treatment phase are administered VT1021. Frequency and dosage are determined in accordance with the stage and severity of disease accordingly to methods standard in the art. Tumor burden and other disease indicators are measured and data compiled using methods known in the art.

It is expected that results will show a reduction in tumor burden in subjects administered VT1021 as compared to control subjects.

The results will show that dually elevated levels of CD36 and CD47 is a robust biomarker for the responsivity of a cancer to treatment with VT1021, including the responsivity of pancreatic cancer. The results will further show that peptides of the present technology, including VT1021, are useful for the treatment of cancers presenting with dually elevated levels of CD36 and CD47 as compared to control levels, including pancreatic cancer.

### Example 4: Treatment of GBM Having Dually Elevated Levels of CD36 and CD47 Using VT1021

This example will demonstrate the efficacy of VT1021 for the treatment of GBM presenting with dually elevated levels of CD36 and CD47.

Subjects having GBM are identified using standard clinical protocols. Samples are obtained and levels of CD36 and CD47 measured using methods known in the art. Subjects presenting with dually elevated CD36 and CD47 levels as compared to suitable controls are selected for dosing with VT1021.

Subjects advancing to the treatment phase are administered VT1021. Frequency and dosage are determined in accordance with the stage and severity of disease accordingly to methods standard in the art. Tumor burden and other disease indicators are measured and data compiled using methods known in the art.

It is expected that results will show a reduction in tumor burden in subjects administered VT1021 as compared to control subjects.

The results will show that dually elevated levels of CD36 and CD47 is a robust biomarker for the responsivity of a cancer to treatment with VT1021, including the responsivity of GBM. The results will further show that peptides of the present technology, including VT1021, are useful for the treatment of cancers presenting with dually elevated levels of CD36 and CD47 as compared to control levels, including GBM.

### REFERENCES

1. Fidler, I. J., The pathogenesis of cancer metastasis: the ‘seed and soil’ hypothesis revisited. Nat Rev Cancer, 2003. 3 (6): p. 453-8.
2. Kang, S. Y., et al., Prosaposin inhibits tumor metastasis via paracrine and endocrine stimulation of stromal p53 and Tsp-1. Proc Natl Acad Sci USA, 2009. 106 (29): p. 12115-20.
3. Lamy, L., et al., Interactions between CD47 and thrombospondin reduce inflammation. Journal of Immunology (Baltimore, Md.: 1950), 2007. 178 (9): p. 5930-9.
4. Salajegheh, M., et al., Upregulation of thrombospondin-1 (TSP-1) and its binding partners, CD36 and CD47, in sporadic inclusion body myositis. J Neuroimmunol, 2007. 187 (1-2): p. 166-74.
5. Vallejo, A. N., et al., Central role of thrombospondin-1 in the activation and clonal expansion of inflammatory T cells. Journal of Immunology (Baltimore, Md.: 1950), 2000. 164 (6): p. 2947-54.
6. Catena, R., et al., Bone marrow-derived Gr1+ cells can generate a metastasis-resistant microenvironment via induced secretion of thrombospondin-1. Cancer Discov, 2013. 3 (5): p. 578-89.
7. Feig, C., et al., The pancreas cancer microenvironment. Clin Cancer Res, 2012. 18 (16): p. 4266-76.

Without further elaboration, one skilled in the art can, based on the above description, utilize the present disclosure to its fullest extent. Embodiments disclosed herein are to be construed as merely illustrative, and not in any way limiting. All publications cited herein are incorporated by reference for the purposes or subject matter referenced herein.

As used herein, indefinite articles “a” and “an,” as used herein in the specification and in the claims, unless clearly indicated to the contrary, should be understood to mean “at least one.”

What is claimed is:

1. A method for evaluating the responsiveness of a subject to cancer treatment with a Tsp-1-inducing agent, the method comprising determining levels of CD36 and CD47 in a sample obtained from the subject, wherein dually elevated levels of CD36 and CD47 in the sample compared to control levels indicates that the subject is or is likely to be responsive to cancer treatment with a Tsp-1-inducing agent.

2. The method of claim 1, wherein the levels of CD36 and CD47 in the sample are determined in vitro.

3. The method of claim 1 or 2, wherein the method further comprises selecting the subject with dually elevated levels of CD36 and CD47 in the sample compared to control levels for treatment with a Tsp-1-inducing agent.

4. The method of claim 3, wherein the method further comprises administering to the subject an effective amount of a Tsp-1-inducing agent to treat the cancer.

5. A method for treating a subject with cancer, the method comprising determining levels of CD36 and CD47 in a sample obtained from the subject, wherein dually elevated levels of CD36 and CD47 in the sample compared to control levels indicates that the subject is or is likely to be responsive to cancer treatment with a Tsp-1-inducing agent, and further comprising administering to a subject having dually elevated levels of CD36 and CD47 in the sample compared to control levels an effective amount of a Tsp-1-inducing agent to treat the cancer.

6. A method for treating a subject with cancer, the method comprising (a) selecting a subject with cancer on the basis that the subject is known to have dually elevated levels of CD36 and CD47 in a sample compared to control levels; and (b) administering to the subject an effective amount of a Tsp-1-inducing agent to treat the cancer.

7. The method of any one of claims 1-6, wherein the control levels are CD36 and CD47 levels in a non-cancerous cell or tissue obtained from the subject.

8. The method of any one of claims 1-6, wherein the control levels are CD36 and CD47 levels in a cell or tissue obtained from a healthy subject or a population of healthy subjects.

9. The method of any one of claims 1-6, wherein the control levels are predetermined levels.

10. The method of any one of claims 1-9, wherein the levels of CD36 and CD47 comprise CD36 and CD47 protein levels a CD36 and CD47 mRNA levels.

11. The method of any one of claims 1-10, wherein the cancer is prostate cancer, breast cancer, ovarian cancer, lung cancer, leukemia, pancreatic cancer, glioblastoma multiforme, astrocytoma, or melanoma.

12. The method of any one of claims 1-11, wherein the Tsp-1-inducing agent comprises a Psap peptide having the amino acid sequence CDWLPK (SEQ ID NO 1), DWLPK (SEQ ID NO 2), or DWLP (SEQ ID NO 3), or an amino acid substitution variant thereof, wherein the amino acid substitution is:
a) Tyrosine (Y) for Tryptophan (W);
b) an amino acid substitution for Leucine (L) selected from Valine (V), Alanine (A) or Glycine (G), or a non-canonical amino acid of similar size, or a derivative thereof; c) Arginine (R) for Lysine (K);
d) a D-isomer of Aspartic Acid (D) for an L-isomer of Aspartic Acid (D) and/or a D-isomer of Leucine (L) for a L-isomer of Leucine (L);
e) a D-isomer of Tryptophan (W) for an L-isomer of Tryptophan (W) and/or a D-isomer of Proline (P) for an L-isomer of Proline (P); or combinations thereof.

13. The method of claim 12, wherein the Psap peptide is 50 amino acids or fewer in length.

14. The method of claim 13, wherein the Psap peptide is 30 amino acids or fewer in length.

15. The method of claim 14, wherein the Psap peptide is 15 amino acids or fewer in length.

16. The method of claim 15, wherein the Psap peptide is 6 amino acids or fewer in length.

17. The method of claim 12, wherein the Psap peptide is a cyclic peptide.

18. The method of any one of claims 12-17, wherein the non-canonical amino acid of similar size is methylvaline, methylleucine, or sarcosine.

19. The method of claims 1-12, wherein the Tsp-1-inducing agent is cyclic DWLPK (SEQ ID NO 2).

20. A composition for use in treating a subject with cancer characterized by dually elevated levels of CD36 and CD47 in a sample compared to control levels, the composition comprising a Tsp-1-inducing agent.

21. Use of a composition for in the manufacture of a medicament for treating a subject with cancer characterized by dually elevated levels of CD36 and CD47 in a sample compared to control levels, the composition comprising a Tsp-1-inducing agent.

22. The composition or use of claim 20 or 21, wherein the control levels are CD36 and CD47 levels in a non-cancerous cell or tissue obtained from the subject.

23. The composition or use of claim 20 or 21, wherein the control levels are CD36 and CD47 levels in a cell or tissue obtained from a healthy subject or a population of healthy subjects.24. The composition or use of claim 20 or 21, wherein the control level is a predetermined level.

24. The composition or use of any one of claims 20-23, wherein the levels of CD36 and CD47 comprise CD36 and CD47 protein levels a CD36 and CD47 mRNA levels.

25. The composition or use of any one of claims 20-24, wherein the cancer is prostate cancer, breast cancer, ovarian cancer, lung cancer, leukemia, pancreatic cancer, glioblastoma multiforme, astrocytoma, or melanoma.

26. The composition or use of any one of claims 20-25, wherein the Tsp-1-inducing agent comprises a Psap peptide having the amino acid sequence CDWLPK, DWLPK, or DWLP, or an amino acid substitution variant thereof, wherein the amino acid substitution is:
a) Tyrosine (Y) for Tryptophan (W);
b) an amino acid substitution for Leucine (L) selected from Valine (V), Alanine (A) or Glycine (G), or a non-canonical amino acid of similar size, or a derivative thereof; c) Arginine (R) for Lysine (K);
d) a D-isomer of Aspartic Acid (D) for an L-isomer of Aspartic Acid (D) and/or a D-isomer of Leucine (L) for a L-isomer of Leucine (L);
e) a D-isomer of Tryptophan (W) for an L-isomer of Tryptophan (W) and/or a D-isomer of Proline (P) for an L-isomer of Proline (P); or combinations thereof.

27. The composition or use of claim 26, wherein the Psap peptide is 50 amino acids or fewer in length.

28. The composition or use of claim 27, wherein the Psap peptide is 30 amino acids or fewer in length.

29. The composition or use of claim 28, wherein the Psap peptide is 15 amino acids or fewer in length.

30. The composition or use of claim 29, wherein the Psap peptide is 6 amino acids or fewer in length.

31. The composition or use of claim 26, wherein the Psap peptide is a cyclic peptide.

32. The composition or use of any one of claims 26-31, wherein the non-canonical amino acid of similar size is methylvaline, methylleucine, or sarcosine.

33. The composition or use of claims 20-26, wherein the Tsp-1-inducing agent is cyclic DWLPK (SEQ ID NO 2).

34. The method, use or composition of any of claims 1-33, wherein the sample is a tumor sample.
